(19) United States
(12) Patent Application Publication
(10) Pub. No.: US 2022/0387620 A1
(43) Pub. Date: Dec. 8, 2022

(54) NANOSTRUCTURED DRUG DELIVERY SYSTEM AS A MULTIFUNCTIONAL PLATFORM FOR THERAPY

(71) Applicant: UNIVERSIDADE DE COIMBRA, Coimbra (PT)

(72) Inventors: Carla Sofia PINHEIRO VITORINO, Coimbra (PT); Alberto António CARIA CANELAS PAIS, Coimbra (PT); João José MARTINS SIMÕES DE SOUSA, Coimbra (PT); Amílcar CELTA FALCÃO RAMOS FERREIRA, Cantanhede (PT); Ana Critina BAIRRADA FORTUNA, Coimbra (PT); Tânia FIRMINO GUERRA GUERREIRO COVA, Coimbra (PT); Sandra Cristina DA CRUZ NUNES, Coimbra (PT); João Diogo BASSO DA SILVA TORRES, Cantanhede (PT); António José LEITÃO DAS NEVES ALMEIDA, Lisboa (PT); Maria Manuel FELICIANO DA COSTA MENDES, Coimbra (PT); LÍdia Maria DIOGO GONÇALVES, Oeiras (PT)

(21) Appl. No.: 17/780,986

(22) PCT Filed: Dec. 3, 2020
(86) PCT No.: PCT/IB2020/061452

§ 371 Date: May 29, 2022

(30) Foreign Application Priority Data
Dec. 3, 2019 (PT) 115950

(51) Int. Cl. A61K 47/69; A61K 47/61; A61K 47/54; A61K 47/64; A61K 31/12; A61K 31/40; A61P 35/00; A61K 47/24

(52) U.S. Cl. CPC A61K 47/6911; A61K 47/61; A61K 47/545; A61K 47/6435; A61K 47/64; A61K 31/12; A61K 31/40; A61P 35/00; A61K 47/24

(57) ABSTRACT

The present invention discloses a drug delivery system for targeted therapy comprising a functionalized lipid-based nanoplatform, where at least one ligand is coupled to the surface of the nanoplatform and encapsulates at least one pharmaceutically active ingredient. A process for obtaining the drug delivery system of the present invention is also disclosed as well as a composition comprising the system of the invention. The invention relates to the field of medicine and biotechnology. The present solution aims at developing targeting co-encapsulating nanostructured lipid carriers for the treatment of different types of cancer, including glioblastoma, as well as other diseases and disorders, envisioning the establishment of an in vitro/in vivo correlation.

### TECHNICAL FIELD

The invention relates to the field of medicine and biotechnology. The present solution aims at developing targeting co-encapsulating nanostructured lipid carriers for the treatment of different types of cancer, including glioblastoma, as well as other diseases and disorders, envisioning the establishment of an in vitro/in vivo correlation.

### BACKGROUND

Cancer remains the one of the leading causes of death globally, as recurrent forms commonly appear after surgical resection thus impairing the complete remission of the tumor. Glioblastoma Multiforme, now referred solely as glioblastoma (GB) is considered the most aggressive and lethal type of brain tumor, accounting for more than half of malignant gliomas (the most common group of primary brain tumors).

Glioblastoma is deeply embedded in the scientific community within the diffuse astrocytic tumor category. In fact, it comprises a group of grade IV lesions caused predominantly by astrocytic cell differentiation, presenting nuclear atypia, cellular pleomorphism, diffuse growth pattern, mitotic activity, microvascular proliferation and with or without necrosis. It is associated with a poor prognosis due to its location and noteworthy diffuse infiltrative characteristics on neighbouring brain structures. Moreover, some of lower World Health Organization (WHO) grade malignant gliomas can recur, progress, or transform into GBs, being termed secondary GBs (over 10% of diagnosed GB cases). The remaining 90% of diagnosed GB cases are primary GBs, also known as de novo GB tumors [1].

Despite the intensive scientific study of the disease, the approved therapy options lack the ability to completely remove all tumor cells. Therefore, GB cannot be cured at present. The standard therapy for GB includes surgical tumor resection, followed by radiotherapy plus concomitant chemotherapy with temozolomide (TMZ), followed by adjuvant TMZ. Moreover, other chemotherapeutic drugs, such as cisplatin, carboplatin, doxorubicin, procarbazine, carmustine, lomustine and bevacizumab have also been considered. However, according to comparative studies involving several chemotherapeutic agents, TMZ provided the highest median survival time in patients [2].

The overall survival time for a patient diagnosed with primary GB and submitted to surgery, radiotherapy and chemotherapy is only of 15 months vs. 9.9 months, if no chemotherapeutic agent is used. In case of the secondary GB, the mean survival time is higher, with 31 months for the triple therapy vs. 24 months for surgery and radiotherapy [1].

The clinical failure of many therapeutic approaches in GB can be due to several factors. On one hand, GB presents a high proliferative activity with infiltration into the surrounding tissues, since it is among the most highly vascularized tumors. This fact does not allow a complete resection of the tumor through surgery, and radiotherapy alone is not always efficient due to the presence of tumor cells in hypoxic areas, which are resistant to radiation. On the other hand, GBs are difficult to treat due to the presence of the natural barrier in the brain, the blood-brain barrier (BBB). Such barrier is known by the tight structure of its endothelial cell layer and the lack of fenestrae, which prevent many therapeutic agents from reaching the brain [1, 3].

With regards to TMZ, despite its ability to cross the BBB and its promising overall efficacy in GB treatment, it requires high systemic doses to reach therapeutic levels in the brain, due to its short half-life. Although generally well-tolerated, there are evidences that TMZ can cause nerve damage, nausea, hair loss, vomiting, infertility, diarrhea, skin rash, myelosuppression, irreversible thrombocytopenia, aplastic anaemia, and even death, raising some concerns about its use. Other factors, including resistance mechanisms, absence of specificity, and poor drug accumulation in tumors, make the conventional treatment for GB limited and with low potential for a clinic use. Inter- and intra-tumor heterogeneity, deregulated signalling pathways, DNA repair pathways, the persistence of cancer stem cells sub-populations and autophagy mechanisms, have been some of the causes associated to TMZ resistance [3].

Despite multiple efforts over the past decades to develop new strategies to treat GB, none of them led to a better prognostic or an enhanced quality of life, when compared to the current standard of care. The treatment of GB remains a big challenge and has been one of the priorities in neuroscience, in order to provide a good prognosis and positive response in GB patients [3].

Bearing in mind the diffuse nature of GB, drug sponsors are increasingly focused on the development of new strategies to kill neoplastic cells far from the tumor bulk, in order to achieve complete tumor eradication. Innovative therapeutic approaches are emerging, including personalized medicine approaches, and novel empirical designs are already being tested in clinical trials. Such therapeutic advances (focusing for instance in in situ drug delivery, hyperthermia therapy, immunotherapy, gene therapy and nanomedicine) are intended to be used in combination with the current standard of care in GB, so as to enhance its therapeutic effect [3].

Nanotechnology has opened doors for potential applications in the field of oncology. Nanoparticle (NP)-based carriers have been intensively explored in an attempt to improve the bioavailability of therapeutic molecules for brain uptake and their tumor targeted delivery. Due to their potential for active/passive targeting, NPs have been considered one of the most appealing drug delivery systems to overcome the limitations of the current treatment. Active targeting takes advantage of the receptors generally overexpressed in certain tumor cells, and not expressed by healthy cells. Affinity ligands (antibodies, peptides, or aptamers) are then capable of binding to antigens or receptors on the target cells, leading to an increased internalization and therapeutic effect [4].

Despite all advantages of NPs, their ability to cross various biological barriers and improve cellular uptake can also cause unexpected toxicity. Actually, NPs also raise safety issues, greatly due to their physicochemical properties (composition, size, surface area and shape). Developing the optimal NP for targeted drug delivery is thereby a constant challenge [4].

Although there is a large number of successful preclinical studies with NPs for GB, their translation to clinical trials has only been partially mastered.

Lipid NPs have attracted attention as promising alternatives to overcome the limitations of traditional polymeric NPs. The first generation of LNPs, also known as solid lipid nanoparticles (SLNs), emerged in the early 1990s as an alternative carrier system to liposomes, emulsions, and polymeric NPs. By definition, SLNs are colloidal particles exhibiting sizes comprised between 40 and 1000 nm, are composed of a biocompatible and biodegradable lipid matrix (solid at room and body temperatures) and stabilized by a suitable surfactant. At the end of the 1990s, nanostructured lipid carriers (NLCs) were introduced as the second generation of solid lipid NPs, aiming at minimizing some inherent limitations of SLNs. They exhibit a lipid matrix with a special nanostructure consisting of both solid and liquid (oil) lipids at room and body temperatures. Due to their less organized lipid core, NLCs present important advantages over SLNs: improved drug loading capacity; reduced polymorphic transition/low crystallinity index and increased stability during storage [4].

The composition of NLCs comprises a set of variables that affect their properties, such as particle size and distribution, and zeta potential. In turn, these properties influence drug loading capacity, encapsulation efficiency, drug release kinetics and stability. The type of surfactant/co-surfactants also influence the applicability of lipid NPs and administration routes. In addition to their distinct applications, lipid NPs are also versatile in terms of loading, as they can encapsulate both lipophilic and hydrophilic drugs, genes, proteins and peptide molecules.

These facts are disclosed in order to illustrate the technical problem addressed by the present disclosure.

### SUMMARY OF THE INVENTION

The present invention discloses a drug delivery system for targeted therapy comprising a functionalized lipid-based nanoplatform, where at least one ligand is coupled to the surface of the nanoplatform and encapsulates at least one pharmaceutically active ingredient.

In an embodiment, the drug delivery system is for brain tumor therapy, wherein the ligand is a tumor-specific ligand.

In a further embodiment, the drug delivery system is for treatment of brain tumors, particularly glioblastoma.

In a further embodiment, the present invention discloses a drug delivery system wherein the lipid-based nanoplatform is a lipid matrix comprising a lipid content of 5-20% (w_{lipid}/wₜₒₜₐₗ).

In a further embodiment, the present invention discloses a drug delivery system wherein the lipid-based nanoplatform comprises a liquid lipid fraction and a solid lipid fraction.

In a further embodiment, the present invention discloses a drug delivery system wherein the solid lipid fraction:liquid lipid fraction ratio is from 25:75 to 75:25.

In a further embodiment, the present invention discloses a drug delivery system wherein the solid lipid fraction comprises cetyl palmitate.

In a further embodiment, the present invention discloses a drug delivery system wherein the nanoplatform comprises one or more solid lipids selected from: cetyl palmitate, stearic acid, glyceryl tripalmitate, cetyl alcohol, cetostearyl alcohol, mono-, di-, triglyceride esters of fatty acids C10-C18, mono-, di-, triesters of C16-C22 acids or a combination thereof.

In a further embodiment, the present invention discloses a drug delivery system wherein the nanoplatform comprises one or more liquid lipids selected from: caprylocaproyl macrogol-8 glycerides, diethylene glycol monoethyl ether, propylene glycol mono-, diesters of C8-C12 acids, medium-chain mono-, di-, triglycerides of C8-C10, monounsaturated omega-9 octadecenoic fatty acid, C30 isoprenoid hydrocarbon, hydrogenated C30 hydrocarbon or a combination thereof.

In a further embodiment, the present invention discloses a drug delivery system wherein the nanoplatform is a lipid matrix additionally comprising a non-ionic surfactant.

In a further embodiment, the present invention discloses a drug delivery system wherein the non-ionic surfactant is one or more selected from: fat free soybean phospholipids, phosphatidylcholine, polysorbate 80, polysorbate 60, polysorbate 40, polysorbate 20, soybean phosphatidylcholine, soybean hydrogenated phosphatidylcholine, polyethylene glycol (15)-hydroxystearate, polyoxyl 40 hydrogenated castor oil, purified polyoxyl 35 castor oil or a combination thereof.

In a further embodiment, the present invention discloses a drug delivery system wherein the non-ionic surfactant is fat free soybean phospholipids at 0.5-3% (w/w), polysorbate 80 at 1-5% (w/w) or a combination thereof.

In a further embodiment, the present invention discloses a drug delivery system further comprising a cationic surfactant.

In a further embodiment, the present invention discloses a drug delivery system wherein the cationic surfactant is octadecylamine.

In a further embodiment, the present invention discloses a drug delivery system wherein the cationic surfactant is one or more selected from: cetrimonium bromide, dodecyltrimethylammonium bromide, didodecyldimethylammonium bromide, dodecyl pyridinium chloride or a combination thereof.

In a further embodiment, the present invention discloses a drug delivery system wherein the mean particle size of the lipid-based nanoplatform is lower than 200 nm, preferably 50 to 150 nm.

In a further embodiment, the present invention discloses a drug delivery system wherein the nanoplatform has a monodisperse size distribution with a polydispersity index lower than 0.3, preferably from 0.1 to 0.3.

In a further embodiment, the present invention discloses a drug delivery system wherein the nanoplatform comprises a nanostructured lipid carrier.

In a further embodiment, the present invention discloses a drug delivery system wherein the nanostructured lipid carrier comprises at least one pharmaceutically active ingredient.

In a further embodiment, the present invention discloses a drug delivery system wherein the pharmaceutically active ingredient is encapsulated, entrapped or intercalated in the nanostructured lipid carrier.

In a further embodiment, the present invention discloses a drug delivery system wherein the pharmaceutically active ingredient is one or more selected from the group consisting of alkylating drugs; cytotoxic antibiotics; platinum drugs; antiparasitics; anti-infectives; drugs acting on nervous system; drugs acting on cardiovascular system; drugs acting on blood; drugs acting on alimentary tract and metabolism; drugs acting on genitourinary system and sex hormones; drugs acting on musculoskeletal system; drugs acting on respiratory system, dermatological drugs or a combination thereof.

In a further embodiment, the present invention discloses a drug delivery system wherein the alkylating drug is one or more selected from: temozolomide, chlorambucil, melphalan, busulfan, lomustine, carmustine, estramustine, cyclophosphamide, chlormethine, treosulfan, mitobronitol or a combination thereof.

In a further embodiment, the present invention discloses a drug delivery system wherein the cytotoxic antibiotic is one or more selected from: doxorubicin, epirubicin, idarubicin, daunorubicin, aclarubicin, dactinomycin, mitomycin, bleomycin, mitoxantrone or a combination thereof.

In a further embodiment, the present invention discloses a drug delivery system wherein the platinum drug is one or more selected from: cisplatin, carboplatin, oxaliplatin or a combination thereof.

In a further embodiment, the present invention discloses a drug delivery system wherein the antiparasitic is one or more selected from: chloroquine, hydroxychloroquine, mefloquine, quinacrine, pyrvinium pamoate, mebendazole or a combination thereof.

In a further embodiment, the present invention discloses a drug delivery system wherein the anti-infective is one or more selected from: acyclovir, valganciclovir, lopinavir, ritonavir, nelfinavir, atazanavir, ribavirin, itraconazole, ciprofloxacin, salinomycin, minocycline, doxycycline, tigecycline, chloramphenicol or a combination thereof.

In a further embodiment, the present invention discloses a drug delivery system wherein the drug acting on nervous system is one or more selected from: chlorpromazine, thioridazine, fluphenazine, perphenazine, olanzapine, penfluridol, quetiapine, lithium, donepezil, memantine, paroxetine, fluoxetine, sertraline, fluvoxamine, imipramine, amitriptyline, clomipramine, doxepin, citalopram, escitalopram, levetiracetam, valproic acid, propofol, disulfiram, dimethyl fumarate or a combination thereof.

In a further embodiment, the present invention discloses a drug delivery system wherein the drug acting on cardiovascular system selected from: digitoxin, atorvastatin, lovastatin, simvastatin, mevastatin, fluvastatin, cerivastatin, pitavastatin, verapamil, mibefradil, losartan, captopril, carvedilol or a combination thereof.

In a further embodiment, the present invention discloses a drug delivery system wherein the drug acting on blood is ticlopidine.

In a further embodiment, the present invention discloses a drug delivery system wherein the drug acting on alimentary tract and metabolism is one or more selected from: metformin, repaglidine, pioglitazone, rosiglitazone, ciglitazone, phenformin, sulfasalazine, aprepitant, cimetidine or a combination thereof.

In a further embodiment, the present invention discloses a drug delivery system wherein the drug acting on genito urinary system and sex hormones is one or more selected from: estradiol, sildenafil or a combination thereof.

In a further embodiment, the present invention discloses a drug delivery system wherein the drug acting on musculoskeletal system and sex hormones is one or more selected from: auranofin, celecoxib or a combination thereof.

In a further embodiment, the present invention discloses a drug delivery system wherein the drug acting on respiratory system is one or more selected from: ibudilast, amlexanox or a combination thereof.

In a further embodiment, the present invention discloses a drug delivery system wherein the dermatological drug is one or more selected from: isotretinoin, ivermectin or a combination thereof.

In a further embodiment, the present invention discloses a drug delivery system wherein the ligand is selected from: hyaluronic acid, folic acid, cRGDfK peptide, H7k(R2)2 peptide or a combination thereof.

In a further embodiment, the present invention discloses a drug delivery system wherein folic acid, cRGDfK peptide and H7k(R2)2 peptide are coupled to hyaluronic acid.

In a further embodiment, the present invention discloses a drug delivery system wherein the targeting ligand delivery system has high binding selectivity for CD44 receptors.

In a further embodiment, the present invention discloses a drug delivery system wherein the targeting ligand delivery system has high binding selectivity for folate receptors.

In a further embodiment, the present invention discloses a drug delivery system wherein the targeting ligand delivery system has high binding selectivity for αvβ3 and αvβ5 integrins.

In a further embodiment, the present invention discloses a drug delivery system wherein the targeting ligand delivery system has high binding selectivity and is tumor-specific pH-responsive.

In a further embodiment, the present invention discloses a drug delivery system for targeting a molecule overexpressed in tumor cells and/or tumor blood vessels.

In a further embodiment, the present invention discloses a process for obtaining the drug delivery system comprising the steps of high shear homogenization followed by ultrasonication or high-pressure homogenization.

In a further embodiment, the invention relates to intravenous composition comprising the drug delivery system of the present invention.

In a further embodiment, the invention relates to a drug delivery system, wherein the nanoplatform surface functionalization with targeting ligands enables specific binding to receptors overexpressed on glioblastoma cells and endothelial cells existing in the blood brain barrier.

In a further embodiment, the invention relates to a drug delivery system, wherein the nanoplatform surface functionalization of the nanoplatform enables an efficient internalization via endocytosis by U-87 MG cells.

### General Description

In an embodiment, the proposed invention provides a nanotechnological based platform, particularly an NLC with a particle size lower than 150 nm, encapsulating a combination of agents for cancer therapy. This is achieved through, but not limited to coupling tumor specific ligands on the surface of the nanosystem to preferentially and/or specifically target cell populations such as tumor cells. Thus, the platform is able, but not limited, to preferentially deliver therapeutic and/or diagnostic agents, alone or in combination, through the BBB to the brain, at the intracellular level, by receptor-mediated endocytosis. This system mediates efficient intracellular release of the encapsulated agents, increasing the concentration of the payload at the target site and reducing agent-associated adverse side-effects. Consequently, the strategy adopted by the invention promotes an improvement of the safety and efficiency for cancer therapy and/or diagnostic.

In a further embodiment, the present invention provides a delivery system able to encapsulate and maintain over time a synergistic or non-synergistic drug combination. Furthermore, the present invention is highly versatile, as it may be subjected to modifications regarding its composition, encapsulated agents or the attached ligands, depending on its purpose (treatment and/or diagnosis, but not limited, of GB and other cancer types or other diseases, as well as other fields of application). The system is functionalized on the surface with one or more internalizing targeting agents, enabling specific receptor-mediated endocytosis into tumor cells, including but not limited to GB cells.

In another embodiment, the proposed prototype was produced through high shear homogenization, followed by ultrasonication (or high-pressure homogenization as alternative technique), using cetyl palmitate, diethylene glycol monoethyl ether, caprylocaproyl macrogol-8 glycerides as lipids, polysorbate 80 and fat free soybean phospholipids (70% phosphatidylcholine) as surfactants, and loaded with curcumin and atorvastatin calcium as model therapeutic agents. In addition, the surface of the nanoplatform was modified with octadecylamine (S) and several targeted ligands, via electrostatic binding to octadecylamine, including hyaluronic acid (HA), folic acid (FA), cyclic arginine(R)-glycine(G)-aspartate(D) peptide (cRGDfK) and H7k(R₂)₂ peptide.

Despite the recent publication of a NLC (please see ref. [5]), also aiming at the treatment of glioblastoma, the object of the present invention displays several and distinct differences in terms of lipid composition and surface modification.

It should be noted that the proposed invention aims at developing and validating a nanotechnological based platform that could fit other therapeutic unmet needs as future in vivo “personalized” medicine applications. Thus, as alternative strategies, other therapeutic drugs, diagnostic agents, genetic material and targeted diseases could be addressed. In addition to the example described in the summary of the invention, the present nanoplatform could serve other purposes, namely, within the Cosmetic, Food Industry and Environmental Engineering. Although the present prototype is suitable for intravenous administration, other administration routes are also viable, such as, oral, topical, intranasal, parenteral, pulmonary and/or ocular.

### BRIEF DESCRIPTION OF THE DRAWINGS

The following figures provide preferred embodiments for illustrating the disclosure and should not be seen as limiting the scope of invention.

FIG. 1. In vitro release profiles of curcumin (A) and atorvastatin calcium (B), co-encapsulated in nanostructured formulations, not modified (F0) and functionalized according to surface modification with octadecylamine (0.16%, w/w) (F1) and HA-based conjugates HA-cRGDfK-H7k(R₂)₂+HA-FA (1:1) (F5). Results are related to predetermined intervals (0.5, 1, 3, 6, 9, 12, 24, 30, 36, 48, 54, 60 and 72 h) and are expressed as mean±SEM (n=6). These formulations are composed by cetyl palmitate, caprylocaproyl macrogol-8 glycerides, diethylene glycol monoethyl ether, polysorbate 80 (5%, w/w), fat free soybean phospholipids (70% phosphatidylcholine) (1%, w/w), atorvastatin calcium (5%, w/w) and curcumin (2.5%, w/w).

FIG. 2. (A) Monitoring of the cytotoxic effect on GB cells of co-encapsulated atorvastatin calcium and curcumin in nanostructured formulations according to surface modification with octadecylamine (0.16%, w/w) (F1) and HA-based conjugates HA-cRGDfK-H7k(R₂)₂+HA-FA (1:1) (F5) over U-87 MG cells. Data is expressed as mean±SD (n=3). ^{a}p<0.05 vs. unloaded-F0 at 24 h; ^{b}p<0.05 vs. unloaded-F0 at 72 h; ^{c}p<0.05 vs. corresponding F5 at 24 h. (B) Monitoring of the cytotoxic effect on non-GB cells of co-encapsulated atorvastatin calcium and curcumin in NLCs according to surface modification with octadecylamine (0.16%, w/w) (F1) and HA-based conjugates HA-cRGDfK-H7k(R₂)₂+HA-FA (1:1) (F5) over THP-1 cells. Data is expressed as mean±SD (n=3).

FIG. 3. (A) In vitro cell uptake of NLC formulations co-encapsulating atorvastatin calcium and curcumin, according to surface modification with octadecylamine (0.16%, w/w) (F1) and HA-based conjugates HA-cRGDfK-H7k(R₂)₂+HA-FA (1:1) (F5) by U-87 MG cells. Data is expressed as mean±SD (n=3, ^{a}p<0.05 vs F0). (B) In vitro hemolytic activity of NLC formulations co-encapsulating atorvastatin calcium and curcumin, according to surface modification with octadecylamine (0.16%, w/w) (F1) and HA-based conjugates HA-cRGDfK-H7k(R₂)₂+HA-FA (1:1) (F5) and considering a range of concentration of atorvastatin calcium (1-100 μg/mL). The assay was conducted with at least 3 different blood donors (n=3), with results being displayed as mean±SD (^{b}p<0.05 vs F0).

FIG. 4. (A) Time-variations of curcumin and atorvastatin in plasma, co-encapsulated in nanostructured formulations according to surface modification with octadecylamine (0.16%, w/w) (F1) and HA-based conjugates HA-cRGDfK-H7k(R₂)₂+HA-FA (1:1) (F5), and in solution (3.75 mg/mL of atorvastatin calcium and 1.875 mg/mL of curcumin in polyethylene glycol 400:saline, 1:1) from to until t₄ h. (B) Biodistribution of atorvastatin calcium and curcumin in mice tissues after administration at 0.5 h with F0, F1 and control. Results are expressed as mean±SEM (4≤n≤6, * not detected).

FIG. 5. Drug selectivity indices (A) and drug targeting indices (B) of co-encapsulated atorvastatin calcium and curcumin in nanostructured formulations according to surface modification with octadecylamine (0.16%, w/w) (F1) and HA-based conjugates HA-cRGDfK-H7k(R₂)₂+HA-FA (1:1) (F5), and in solution (3.75 mg/mL of atorvastatin calcium and 1.875 mg/mL of curcumin in polyethylene glycol 400:saline, 1:1) in brain, liver and spleen tissues.

FIG. 6. (A) Weekly tumor diameter and volume (B) measurements by MR Imaging of experimental (co-encapsulated atorvastatin calcium and curcumin in a surface modified nanostructured formulation with octadecylamine (0.16%, w/w) and HA-based conjugates HA-cRGDfK-H7k(R₂)₂+HA-FA (1:1) (F5) and non-encapsulated drugs in solution) and control groups. Data are represented by mean±SEM (4≤n≤5).

FIG. 7. (A) Body weight variation of daily administered mice with co-encapsulated atorvastatin calcium and curcumin in a surface modified nanostructured formulation with octadecylamine (0.16%, w/w) and HA-based conjugates HA-cRGDfK-H7k(R₂)₂+HA-FA (1:1) (F5) (6≤n≤14), non-encapsulated drugs in solution or with saline, at 0, 7, 14 and 21 days. (B) Serum quantification of aspartate aminotransferase (AST), alanine aminotransferase (ALT) and alkaline phosphatase (ALP) of tumor bearing mice upon euthanasia, treated with co-encapsulated atorvastatin calcium and curcumin in a surface modified nanostructured formulation with octadecylamine (0.16%, w/w) and HA-based conjugates HA-cRGDfK-H7k(R₂)₂+HA-FA (1:1) (F5) and non-encapsulated drugs in solution) and saline. Results are expressed as mean±SEM (4≤n≤5, ^{a}p<0.05 vs saline).

### DETAILED DESCRIPTION

In an embodiment, the present invention aims at developing and validating a versatile nanotechnological based system, which combines the ability, but not limited, to encapsulate therapeutic or diagnostic agents and to target specific cells, particularly glioblastoma tumor cells through the coupling of targeting ligands onto its surface that preferentially and/or specifically aim at overexpressed molecules on target cells. When administered to a subject, the suggested system allows the delivery of therapeutic agents, single or in combination, in a controlled release profile, to target cells, particularly glioblastoma tumor cells.

The terms “nanosystem” or “nanoplatform” refer to a physical, chemical or biological material to which the ligand is linked to, either being the nanostructure or enclosing a therapeutic compound or a combination to interact with a target organ or a specific cell. Examples of “nanosystems” include, but are not restricted to polymeric, inorganic or lipid nanoparticles, liposomes, micelles, nanoemulsions, among others. Preferably, the nanosystem that acts as a support is a nanostructured lipid carrier composed of, but not limited to, solid lipids, such as, cetyl palmitate, stearic acid, glyceryl tripalmitate, cetyl alcohol, cetostearyl alcohol, mono-, di-, triglyceride esters of fatty acids (C10-C18), mono-, di-, triesters of C16-C22 acids or a combination thereof, liquid lipids, such as, caprylocaproyl macrogol-8 glycerides, diethylene glycol monoethyl ether, propylene glycol mono-, diesters of C8-C12 acids, medium-chain mono-, di-, triglycerides of C8-C10, monounsaturated omega-9 octadecenoic fatty acid, C30 isoprenoid hydrocarbon, hydrogenated C30 hydrocarbon or a combination thereof, stabilized by surfactants, such as, fat free soybean phospholipids (70% phosphatidylcholine), polysorbate 80, polysorbate 60, polysorbate 40, polysorbate 20, soybean phosphatidylcholine, soybean hydrogenated phosphatidylcholine, polyethylene glycol (15)-hydroxystearate, polyoxyl 40 hydrogenated castor oil, purified polyoxyl 35 castor oil, octadecylamine, cetrimonium bromide, dodecyltrimethylammonium bromide, didodecyldimethylammonium bromide, dodecyl pyridinium chloride or a combination thereof.

The term “encapsulated” refers to the entrapment, encapsulation or intercalation of the therapeutic agents within the nanosystem, stressing that the agent or a combination are entrapped in the inner core of the system.

The terms “therapeutic agent” and “drug” are employed to designate a polymeric or a non-polymeric organic chemical, a nucleic acid or an oligonucleotide, a peptide, protein, antibody, growth factor or a fragment thereof presenting a linear or cyclic conformation, or a nanostructure expected to significantly alter cell function or the status of cells to which it is delivered to. The therapeutic agents considered in the proposed nanosystem are atorvastatin calcium (ATO) and curcumin (CUR). ATO, a statin that inhibits 3-hydroxy-3-methylglutaryl-coenzyme A (HMG-CoA) reductase activity, is widely used as a cholesterol-lowering agent and has demonstrated anti-cancer benefits, being considered a potential agent with anti-tumor activity for the treatment of GB. CUR, a bioactive compound derived from the plant Curcuma longa, presents anti-inflammatory and antioxidant properties and has been used as an anticancer agent for the treatment of GB. Other potential non-cytotoxic drugs have been tested for anticancer properties in GB treatment, covering several pharmacotherapeutic classes. These include antiparasitics, such as chloroquine, hydroxichloroquine, mefloquine, quinacrine, pyrvinium pamoate and mebendazole; anti-infectives, such as acyclovir, valganciclovir, lopinavir, ritonavir, nelfinavir, atazanavir, ribavirin, itraconazole, ciprofloxacin, salinomycin, minocycline, doxycycline, tigecycline and chloramphenicol; drugs acting on nervous system, such as chlorpromazine, thioridazine, fluphenazine, perphenazine, olanzapine, penfluridol, quetiapine, lithium, donepezil, memantine, paroxetine, fluoxetine, sertraline, fluvoxamine, imipramine, amitriptyline, clomipramine, doxepin, citalopram, escitalopram, levetiracetam, valproic acid, propofol, disulfiram and dimethyl fumarate; drugs acting on cardiovascular system such as digitoxin, atorvastatin, lovastatin, simvastatin, mevastatin, fluvastatin, cerivastatin, pitavastatin, verapamil, mibefradil, losartan, captopril and carvedilol; drug acting on blood, such as ticlopidine; drugs acting on alimentary tract and metabolism, such as metformin, repaglidine, pioglitazone, rosiglitazone, ciglitazone, phenformin, sulfasalazine, aprepitant and cimetidine; drugs acting on genito urinary system and sex hormones, such as estradiol and sildenafil; drugs acting on musculoskeletal system, such as auranofin and celecoxib; drugs acting on respiratory system, such as ibudilast and amlexanox; dermatologicals, such as isotretinoin and ivermectin. Yet, as previously referenced, temozolomide, cisplatin, carboplatin, doxorubicin, procarbazine, carmustine, lomustine and bevacizumab are cytotoxic drugs that have also been used, showing promising results for the treatment of GB. According to the method of the invention, a variety of therapeutic agents can be directed to tumor blood vessels and tumor cells in a subject.

The terms “ligand” and “targeting ligand” designate the molecules linked to a support of the nanosystem that provide the capacity to preferentially and specifically direct the nanosystem to a target, but not to non-target cells, promoting its intracellular delivery. The ligand or targeting ligand of the invention is linked to the surface of the nanosystem in order to identify and interact with the target molecule overexpressed on the surface of a specific cell. The ligand or targeting ligand can be, but not limited to, a protein, a growth factor, a peptide, an aptamer, an antibody, a nanobody or a fragment thereof. Ligands and target, in parenthesis, include, but not limited to, transferrin peptide (transferrin receptor), apolipoprotein E (low density lipoprotein receptor), polysorbate-80 (low density lipoprotein receptor), folic acid (folate receptors), angiopep-2 (low density lipoprotein receptor-related protein-1), glucose-cholesterol derivative L (glucose transporter), rabies virus glycoprotein peptide (nicotinic acetylcholine receptor), cationic bovine serum albumin (negatively charged blood-brain barrier membrane) chitosan/chlorotoxin (negatively charged blood-brain barrier membrane/GB cells), H7k(R₂)₂ peptide (acidic pH-tumor microenvironment), activatable cell-penetrating peptide (acidic pH-tumor microenvironment, metalloproteinase 2, vascular endothelial growth factor, urokinase plasminogen activator), peptide-22 (av33 integrin), cyclic arginine-glycine-aspartate peptide (av33 integrin), bevacizumab (vascular endothelial growth factor), K237 peptide (vascular endothelial growth factor receptor 2), PGN63 (phosphatidylserine), hyaluronic acid (CD44), fibrin-binding pentapeptide cysteine-arginine-glutamic acid-lysine-alanine (fibrin deposition), p-hydroxybenzoic acid (av33 integrin), cetuximab (epidermal growth factor receptor, epidermal growth factor receptor variant III) and small interfering RNA molecules (galectin-1, methylated 06-methylguanine-DNA methyltransferase).

The term “tumor” encompasses all related cells present in the tumor microenvironment, including bulk GB cells with limited self-renewal characteristics, GB stem cells with extensive self-renewal and tumorigenic potential, supporting stroma and angiogenic blood vessels that infiltrate the tumor cell mass.

The proposed invention was optimized following a screening using multivariate analysis methods, in particular, Principal Component Analysis (PCA), Partial Least Square (PLS) regression and a two- and three-level full factorial designs. These methods were used to investigate the effect of the surfactants (hydrogenated phosphatidylcholine or fat free soybean phospholipids (70% phosphatidylcholine) with polysorbate 80) and their concentration, and the composition and the solid:liquid lipid ratio on mean particle size (PS), polydispersity index (PI) and zeta potential (ZP) (data not shown). Moreover, high shear homogenization followed by ultrasonication (US) or high-pressure homogenization (HPH) as production methods were also evaluated in terms of colloidal properties and stability (data not shown).

The optimized invention is composed of 5-20% (w/w) of lipid content, in particular, cetyl palmitate, diethylene glycol monoethyl ether and caprylocaproyl macrogol-8 glycerides, in solid:liquid lipid ratios comprised between 25-75:75-25; 0.5-3% (w/w) of the oil phase surfactant fat free soybean phospholipids (70% phosphatidylcholine); 1-5% (w/w) of the aqueous phase surfactant polysorbate 80; 2.5% (w/w) of CUR and 5% (w/w) of ATO. Diethylene glycol monoethyl ether and caprylocaproyl macrogol-8 glycerides were selected to proceed with the investigation and design the suggested invention also based on the favorable solubility of CUR and ATO in these lipids.

The invention is obtained using high shear homogenization followed by ultrasonication. Lipid and aqueous phases are pre-emulsified using a high-speed stirrer (Ultra-Turrax X1020; Ystral GmbH, Dottingen, Germany), followed by ultrasonication (Branson® Sonifier 250; Branson Ultrasonics Corporation, Connecticut, USA). The resulting dispersion is then stored at 4° C. to form the lipid nanosystem. 24 h after the production of the proposed invention, an ultrafiltration purification procedure is conducted in order to remove the excessive amount of free polysorbate 80. The proposed invention is then homogenized, collected and kept stored at 4° C.

High-pressure homogenization technique has also proved to be a viable alternative method for the production of the optimized proposed invention. This technological method is a well-established technique for the preparation of lipid emulsions and is characterized by its reproducibility and facilitated scale-up. Under certain conditions, instead of being submitted to ultrasonication, the pre emulsion may be processed in a pre-heated high-pressure homogenizer (EmulsiFlex-C3; Avestin, Inc., Ottawa, Canada), thus producing a nanosystem with a similar particle size (data not shown).

Example 1 below presents the parameters used to generally characterize nanostructured formulations at the level of their PS, PI and ZP, as NPs critical quality attributes. The most suitable values proposed in the literature for PS and PI of NPs are in the range of 70-200 nm and lower than 0.25, respectively. Therefore, NPs should be preferentially small with a monomodal and monodisperse size distribution. On the other hand, ZP is another important parameter that has a direct impact on long term stability. A ZP higher than 1301 mV is predictive of physical stability, although the use of polymer-based coating strategies may also increase the nanosystem stability.

Positively charged NLCs have a greater ability to interact with the negatively charged lipid membranes, improving their capability to cross the BBB. Therefore, 0.16 (w/w) of octadecylamine is used to reverse the negative charge of the nanosystem, containing 7.5 (w/w) of lipid content, in particular, cetyl palmitate, diethylene glycol monoethyl ether and caprylocaproyl macrogol-8 glycerides in a solid:liquid lipid ratio of 25:75, 1% (w/w) of fat free soybean phospholipids (70% phosphatidylcholine), 5% (w/w) of polysorbate 80, 2.5% (w/w) of CUR and 5% (w/w) of ATO. According to octadecylamine pKa, the manufacturing process is slightly modified, with the replacement of the aqueous solution of 5% (w/w) polysorbate 80 by an acetate buffer solution (pH=4) containing 5% (w/w) polysorbate 80.

The functionalization of the proposed system is performed through the attachment of tumor targeting ligands onto its surface, namely HA, HA-FA, HA-cRGDfK-H7k(R₂)₂ and HA-cRGDfK-H7k(R₂)₂+HA-FA (1:1) conjugates. The functionalized NLCs are designed and prepared in order to evaluate their potential targeting to GB tumor cells and their anti-tumor activity in an orthotopic xenograft animal model. These strategies provide an active targeting co-encapsulated drug delivery system.

HA, a biodegradable, biocompatible and non-immunogenic glycosaminoglycan, has been largely used for tumor targeting. The strategy focuses in the fact that many types of tumor cells overexpress HA receptors (e.g. CD44). However, poor selectivity is evidenced especially due to the fact that CD44 easily saturates. Coupling HA to NPs, including the combinations HA-cRGDfK or HA-cRGDfK-H7k(R₂)2, is an interesting approach that provides a more tumor-specific targeting. Cyclic arginine(R)-glycine(G)-aspartate(D) (cRGD) peptide is a promising ligand due to its high-binding selectivity for αvβ3 and αvβ5 integrins, heterodimeric receptors that mediate tumor growth, metastasis and tumor angiogenesis, which are overexpressed on the endothelial cells of tumor angiogenic vessels, as well as in GB cells (e.g. U-87 MG cell line). Hence, it has been reported that H7k(R₂)2, an arginine(R)-rich peptide that possesses the pH trigger sequence H7, can respond to the acidic pH microenvironment in gliomas tissues due to the ionization of polyHis, switching from hydrophobic to hydrophilic under acid conditions, being considered a tumor-specific pH-responsive peptide. On the other hand, this peptide presents cell-penetrating peptides (CPP) characteristics (sequence (R2)2), which provide the ability to cross the BBB and accumulate in the brain in a seemingly energy independent manner.

FA is a small and stable non-immunogenic water-soluble compound with critical activity over DNA synthesis, methylation and repair. Considering the over expression of folate receptors in several cancer cells, including GB, epithelial, ovarian, cervical, breast, lung, kidney and colorectal, and due to the retention of its activity when conjugated with drugs or other molecules, folic acid presents itself as a potential enhancer of the internalization of anticancer drugs by tumor cells.

The development of triple targeting co-encapsulated NLCs, but not limited to, is an interesting strategy for the treatment of GB, providing the suitable properties to enhance both tumor targeting and anti-tumor activity.

PS, PI and ZP were evaluated after the purification and surface tailoring of the step-by-step modified NLC formulations, codified according to their composition (Table 1). The analysis of the results shows a gradual increase of particle size with the sequential addition of octadecylamine, HA and the developed conjugates (HA-FA, HA-cRGDfK-H7k(R₂)2 and HA-cRGDfK-H7k(R₂)₂+HA-FA (1:1)). Concerning PI, although the polydispersity index increases in F5, DLS measurements indicate the presence of a single peak with an average size of 124.0 nm. As for ZP, it shifts to positive values with the introduction of octadecylamine, but decreases with the subsequent addition of the conjugates, due to the electrostatic interactions of the carboxyl moieties of HA with the amine groups of octadecylamine.

In general, PS tends to increase over time for the formulations modified with octadecylamine and the HA-based conjugates, HA-cRGDfK-H7k(R₂)₂ and HA-FA. However, surface modification improves particle stability. As for ZP, it shows an initial tendency to decrease in the formulation modified with S. For the remaining formulations, it presents the opposite behaviour (data not shown). Note that the formulation F5 presents a lower ZP value over the one containing only octadecylamine (F1), suggesting a successful electrostatic binding of S to HA and subsequent coating of the NPs.

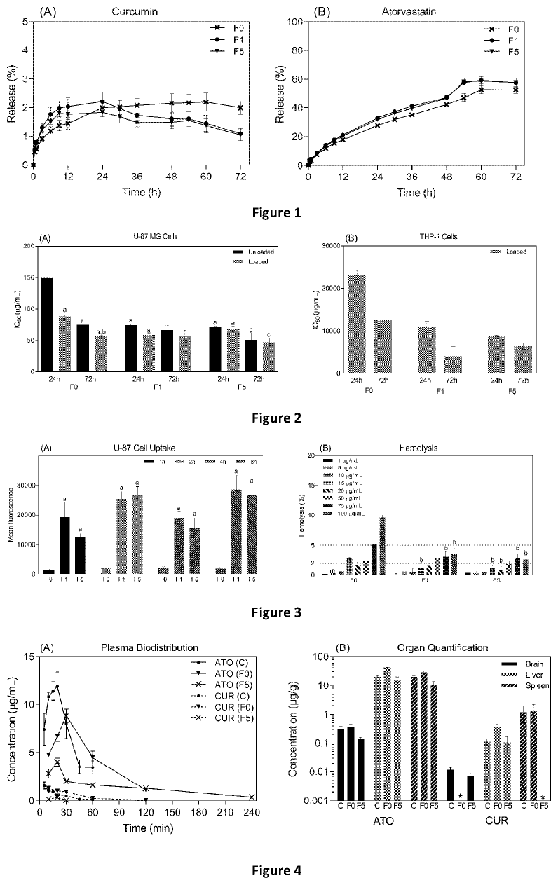

The in vitro drug release from NLCs was explored for surface modified nanosystems (Example 2). According to the release profiles displayed in FIG. 1, NLCs exhibit a sustained drug release for both compounds, with no more than approximately 2 and 60% CUR and ATO, respectively, being quantified in the medium at 72 h, confirming the co-encapsulation within the lipid matrix. However, ATO appears to have a less stable behavior in the NLCs, which may be ascribed to the higher solubility in the release medium. Considering that no significant differences were found between F1 and F5, surface modification with HA-based conjugates does not impact drug release. However, the addition of octadecylamine slightly increases ATO release after 24 h (p<0.05).

In vitro toxicity studies were performed as described in Example 3. To assess the potential cytotoxicity of both loaded and unloaded NPs, the resazurin assay was conducted in U-87 MG cells. The sensitivity of GB cells to free ATO and CUR was assessed following a treatment with a drug solution in the range from 5 to 800 μM and 5 to 400 IM, respectively. After 24 h and 72 h of incubation, both compounds were able to impair cellular proliferation. Note that the growth inhibitory effect was more evident on CUR, thus suggesting a higher sensitivity of U-87 MG cells to this compound. The IC₅₀ values of free ATO and CUR at 24 h were 134±27 μM and 86±35 μM, respectively, whereas at 72 h were found to be 25±5 μM and 77±1 μM, respectively.

The influence of the surface modification strategies, as well as the potential cytotoxicity of the NLCs were evaluated, indicating that, at 24 h, F0 shows a negligible toxicity on U-87 MG cells, when compared to further approaches (FIG. 2). In fact, unloaded NLCs, specifically, unloaded-F1 and unloaded-F5 were more cytotoxic than unloaded-F0. This may be ascribed to the charge inversion with the introduction of octadecylamine, which promotes a better penetration through plasma membranes than anionic particles. Additionally, positively charged NLCs may interact with several anionic conjugates and, considering the overexpression of CD44, FA and cRGD receptors on U-87 MG cells, a stronger inhibitory effect is observed.

The addition of both drugs to the same lipid matrix improved the cytotoxic profile of the NLCs, as loaded nanosystems presented a higher cytotoxicity on cancer cells, when compared to the correspondent unloaded formulations. Taking into consideration the delivery of both drugs, F0 and F5 showed, respectively, at 24 h, a 1.69- and 1.05-fold increase in cell death. At 72 h, drug co-delivery in F0 and F5 promoted a 1.32- and 1.08-fold decrease in cell viability. Moreover, results show a time-dependent cytotoxicity, as the exposition of cells for 72 h leads to a higher cytotoxic effect, most likely due to an improved intracellular release of both drugs, as F0 and F5 present a 1.57- and 1.46-fold increase in cytotoxicity, when compared to the correspondent unloaded nanosystem.

As expected, surface functionalized NLCs (F1 and F5) induce a lower cell viability, when compared to the other formulations under study. Note that the highest toxicity was obtained for F5, at 72 h. Overall, the results demonstrate that the targeting strategies improved the interaction of NLCs with cells and play an important role in the cytotoxicity profile of the nanosystems. This was later confirmed by cellular uptake studies, in which U-87 MG cells were incubated for 1, 2, 4 and 8 hours, at 37° C. in 5% CO₂, with Coumarin 6-loaded NLCs at 20 μg/mL of lipid content, and analyzed by flow cytometry. A parallel assay with THP-1 cells was conducted to exclude a non-targeted and systematic toxicity of the nanosystem. As shown in FIG. 2, the tested nanosystems show a lack of toxicity over these cells, thus indicating the proposed invention does not induce cell death indiscriminately.

A systematic analysis of the cellular internalization mechanisms and pathways of Coumarin 6-loaded NLCs was conducted. Cell related fluorescence was considered not significant and confirmed by flow cytometry. Results in FIG. 3 show that the mean fluorescence after 1 h, 2 h, 4 h and 8 h of incubation with Coumarin 6-loaded NLCs is higher whenever these same nanosystems are functionalized. These results suggest that functionalized NLCs (F1 and F5) are easily and rapidly internalized by U-87 MG cells, in contrast to the uncoated nanosystem. No significant differences were observed between the 2 hours and 8 hours in terms of mean fluorescence values indicating a similar internalization of formulations. A comparison of the time-dependent fluorescence intensity levels indicates that the rate of cell uptake was highest for the cationic/surface modification NLCs (F1˜F5), followed by anionic NLCs (F0). Overall, these results suggest that both positive charge and surface modification play a significant role on cell uptake.

The hemolytic behavior of NLCs was accessed by in vitro incubation with human blood, to mimic the possible interaction of the NPs with erythrocytes. The evaluation of the integrity of the membranes was assessed by the quantification of the released hemoglobin. As expected, Triton™ X-100 led to a complete hemolysis of the cells, which did not occur with the negative control PBS. Moreover, no significant hemolysis (<2%) was detected after the incubation of aqueous and acetate buffered polysorbate 80 at 15 and 100 μg/mL (w/V). The results presented in FIG. 3 show a dose-dependent hemolytic activity for all formulations, with the highest concentrations (amount of NPs corresponding to 75 and 100 μg/mL of ATO) tested for F0 being considered hemolytic, as the % of hemolysis is above 5%. Interestingly, the introduction of octadecylamine 0.16% (w/w) significantly reduced hemolysis (p<0.05), which may be due to the interaction of positive charged NPs with plasma proteins.

The aforementioned in vitro results prompted the administration of F0 and F5 formulations, in order to compare the impact of the active targeting surface bioconjugation over the unmodified (F0) nanosystem. The in vivo studies aimed at characterizing and understanding the pharmacokinetics of ATO and CUR after intraperitoneal administration of F0 and F5 formulations, in comparison to a control containing non-encapsulated compounds in the same concentration (Example 4). Time-variations of plasma and brain, liver and spleen concentrations of ATO and CUR at 0.5 h are plotted in FIG. 4. The corresponding pharmacokinetic parameters are displayed in Table 2 and FIG. 5. Considering the tₘₐₓ values, encapsulated drugs seem to take longer time to attain the bloodstream, when compared to the control. Corroborating these findings are also the plasmatic maximum concentrations for both analytes in solution, which are also higher than those obtained for usNLCs. According to the plasma concentrations, a different magnitude is observed for the two compounds, with ATO/CUR assuming AUCₐii values of 578.25/60.05 min×μg/mL for F0, 300.79/6.98 min×μg/mL for F5 and 417.16/32.41 min×μg/mL for the drug solution. However, despite the lower plasmatic concentrations obtained for usNLCs, brain tissue concentration for ATO and CUR are within the same magnitude, whether they are co-encapsulated in usNLCs or free in solution. Regarding CUR, there is a noteworthy brain exposition for F5, in relation to F0, thus suggesting a preferential targeting ability ascribed to the surface modification of the NLCs. In what concerns ATO, it is evidenced that drug encapsulation favors brain targeting, irrespective of the formulation (F0 or F5) considered. The analysis of the DSIs, which reflect the ratio of the drug content in a determined organ and the one measured in plasma, indicates a higher selectivity of ATO to the brain, when co-encapsulated in usNLCs (0.094 for F0 vs. 0.046 for F5 vs. 0.011 for control), despite having a similar behavior in the liver and spleen. In turn, CUR presents a lower DSI, when co-encapsulated in F0, than in F5 or free in solution (0.005 for F0 vs. 1.525 for F5 vs. 0.047 for control). Note that despite having a higher elimination by the liver and spleen tissues, the selectivity of CUR is also improved by the formulation F5. Taking into account the DTIs, which compare the extent of the drug content after the administration of encapsulated and non-encapsulated formulations in a determined organ, there is a higher targeting effect to the brain for both compounds, as the DTI is higher than 1, with the exception of CUR in F0 (0.182 for F0 vs. 5.957 for F5). As expected, high DTI values were obtained for the compounds in both formulations in the liver (2.011 for F0 and 2.516 for F5, regarding ATO; 3.580 for F0 and 1.960 for F5, regarding CUR), while DTI values lower than 1 in F5 indicate a reduced targeting effect to the spleen (0.815 and 0.253 for ATO and CUR, respectively). In addition, co-encapsulated ATO and CUR in F5 present a lower hepatic and splenic elimination than those in F0, due to lower DTI values, suggesting a slower elimination of surface shaped usNLCs. According to these results, F5 appears to have a preferential targeting behavior to the brain, while simultaneously reducing the elimination by clearance organs (liver and spleen).

The safety and tolerability of the formulation F5 was assessed, since the multiple administrations of foreign objects may impair the safety and well-being of animals. The daily intraperitoneal administration of F5 to mice did not elicit significant changes in their body weight (data not shown). Throughout the study there were no significant changes regarding coat condition, food intake, behavior, presence of loose stools, diarrhea, blood in diarrhea or brain inflammation in both control and study groups. The quantification at 24 h post dosing at day 7, 14 and 21 did not show hepatic or splenic accumulation of ATO, CUR or significant variations in AST, ALT or ALP, suggesting the maintenance of liver and spleen functions (data not shown). Overall, daily administrations of F5 intraperitoneally are considered safe and tolerable by mice.

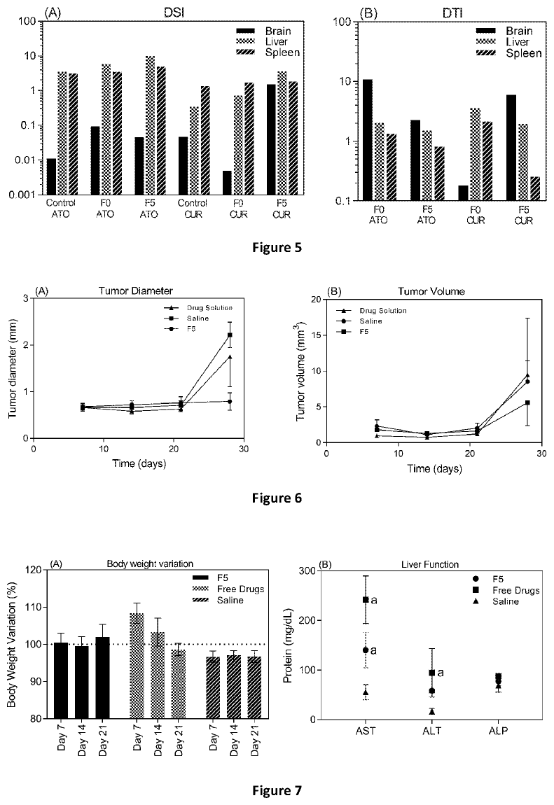

Weekly MR imaging measurements of tumors were conducted in order to evaluate the impact of F5 on tumor size and growth and, consequently, its antitumor efficacy, as presented in Example 5. According to FIG. 6, tumor size remained constant until day 21, with no significant fluctuations for all tested groups. However, from day 21 to day 28, there was a 181% increase in tumor diameter of mice treated with non-encapsulated drugs, while the saline group presented a mean increase of 229%. A similar trend may be observed for tumor volume measurements where, at day 28, the F5 formulation presents a stronger antitumoral performance. The F5 group showed the best results by reducing tumor growth and development in all mice.

The daily intraperitoneal administration of F5 to mice did not elicit significant changes in body weight as depicted in FIG. 7. Interestingly, the administration of the non-encapsulated drugs led to a 8.4% increase in body weight after the first week of treatment. An opposite trend may be observed from day 7 to the last day of the experiment. As for the control group, an initial body weight loss of 3.3% was observed in the first week, with mice maintaining their body weight throughout the remaining days of the study. Furthermore, daily injections of NPs showed a better safety profile than the administration of non-encapsulated drugs. In fact, the co-administration of both compounds in solution led to the major increase in serum levels of AST and ALT, thus indicating acute hepatic toxicity. No significant variations on the levels of serum alkaline phosphatase were detected for formulation F5 (FIG. 7).

### EXAMPLES

The following set of examples is intended to illustrate but not limit the present invention.

### Example 1—Physicochemical Characterization

The mean PS and PI were measured by dynamic light scattering (DLS) using a Zetasizer Nano ZS (Malvern Instruments, Malvern, UK) set at a detection angle of 173° at 25° C. The size of a particle is calculated from the translational diffusion coefficient by using the Stokes-Einstein equation. Cumulants method was used for data analysis. ZP was determined in the same apparatus, set at a temperature of 25° C., according to the Helmholtz-Smoluchowsky equation. Nanodispersions were suitably diluted in ultra-purified water and analyzed three times. The results are represented as mean±standard deviation.

TABLE 1
Physicochemical characteristics, particle size (PS), polydispersity index (PI) and zeta
potential (ZP), of fresh purified drug loaded NLC formulations also encapsulated with
coumarin 6 (1%, w/w).

| | NLC formulation | PS (nm) | PI | ZP (mV) |
|---|---|---|---|---|
| F0 | PTL (25:75) ATO CUR | 46.2 ± 0.3 | 0.180 | −13 ± 2 |
| F1 | PTL (25:75) ATO CUR, S | 57.2 ± 0.4 | 0.171 | 13.8 ± 0.3 |
| F2 | PTL (25:75) ATO CUR, S HA | 83.0 ± 0.3 | 0.118 | 7.8 ± 0.4 |
| F3 | PTL (25:75) ATO CUR, S HA-FA | 82.2 ± 0.6 | 0.128 | 9.1 ± 0.2 |
| F4 | PTL (25:75) ATO CUR, S HA-cRGDfK-H7k(R₂)₂ | 76.4 ± 0.1 | 0.131 | 9.1 ± 0.6 |
| F5 | PTL (25:75) ATO CUR, S HA-cRGDfK-H7k(R₂)₂ + HA-FA (1:1) | 124.0 ± 0.2 | 0.268 | 7.5 ± 0.4 |

P: cetyl palmitate;
T: diethylene glycol monoethyl ether;
L: caprylocaproyl macrogol-8 glycerides;
ATO: atorvastatin calcium;
CUR: curcumin;
S: octadecylamine 0.16% (w/w);
HA: hyaluronic acid;
FA: folic acid,
HA FA: hyaluronic acid-folic acid conjugate;
HA-cRGDfK-H7k(R₂)₂: hyaluronic acid-cyclic arginine(R)-glycine(G)-aspartate(D) peptide-H7k(R₂)₂ peptide conjugate;
PS: particle size;
PI: polydispersity index;
ZP: zeta potential

### Example 2—In Vitro Release Assays

In vitro release studies of purified nanostructured formulations with surface modifications, specifically, octadecylamine 0.16% (w/w) (F1) and octadecylamine 0.16% (w/w) and HA-based conjugates (F5) were performed using a dialysis cellulose membrane (MWCO≈14.000, avg, flat width 33 mm, D9652, Sigma-Aldrich). In order to ensure the sink conditions, the release medium was composed of 30% (V/V) of PEG 400 and 70% (V/V) of phosphate buffered saline (PBS, pH=7.4). Samplings were performed at specific time points, followed by drug quantification through a HPLC validated technique. Release profiles are displayed in FIG. 1.

### Example 3—In Vitro Toxicity Studies

Regarding cell viability, U-87 MG cells were cultured in DMEM/F-12 medium, supplemented with 10% (v/v) FBS, 1% (v/v) penicillin-streptomycin solution and sodium bicarbonate. Cells were maintained at 37° C. in a humidified atmosphere containing CO₂ (5%). The resazurin assay was used to determine the cytotoxicity of NLCs. Briefly, 2000 cells per well were seeded in a 96 well plate and incubated for 24 h or 72 h after the medium was replaced with increasing concentrations of free drugs or NLCs. Subsequently, the medium was removed and the cell viability determined. For this purpose, 100 μL of 10% (w/v) resazurin solution in DMEM/F 12 was added to the cells and incubated for approximately 2 h, at 37° C. The enzymatic reduction of resazurin to resorufin was analyzed by UV/Vis spectrophotometry at 570 nm and 600 nm. Cell viability was assessed indirectly according to equation 1.

$$\% Cell Viability = 100 \times \frac{\text{?}}{\text{?}} \qquad ( eq . 1 )$$

$$\text{?} \text{indicates text missing or illegible when filed}$$

As for THP-1 cells, they were suspended in RPMI medium, supplemented with 10% (v/v) FBS, 1% (v/v) penicillin-streptomycin solution, HEPES and sodium bicarbonate. Cells were maintained at 37° C. in a humidified atmosphere containing CO₂ (5%). The resazurin assay was used to determine the cytotoxicity of NLCs. Briefly, 2000 cells were seeded in a 96 well plate and incubated for 24 h or 72 h after which increasing concentrations of NLCs were added to the medium. Subsequently, cells were centrifuged at 500 g for 5 min and the medium replaced by 100 μL of 10% (w/v) resazurin solution in RPMI was added to the cells and incubated for approximately 4 h, at 37° C. Cell viability was assessed as described above.

All experiments were performed in triplicate and the total lipid content of the particles reflecting a 50% reduction in cell viability (IC₅₀) was determined from the concentration-response curves.

Concerning cell uptake assay NLCs cellular uptake by U-87 MG cells were further studied by flow cytometry (ACEA NovoCyte®, ACEA Biosciences, Inc., USA). For that, 0.5 mL of cell suspension with 100 000 cells per well were seeded 24 hours before the experiments into 24 well tissue culture test plates. The samples of Coumarin 6-loaded NLCs at 20 μg/mL of solid content (0.08 μg/mL of Coumarin 6) were added and incubated for 1 h, 2 h, 4 h and 8 h, at 37° C. in 5% CO2. Free Coumarin 6 was removed by ultrafiltration using centrifugal filter devices (Amicon© Ultra, Ultra cell-50k, Millipore, USA), thereby removing its interference in the measurements. The control group was treated following the same steps without the presence of NLCs. After incubation, cells were washed three times with cold PBS to remove free NLCs and trypsinized. Fluorescence of Coumarin 6 (λexc=485 nm, λem=520 nm) was measured. Then, cells were centrifuged at 1500×g for 5 min and fixed with 4% paraformaldehyde for 30 min at room temperature. Cells were once again washed with PBS, centrifuged, resuspended in PBS and analyzed through flow cytometry. Cell uptake of NLCs by U-87 MG cells is displayed in FIG. 3.

The haemolytic behaviour of NLCs formulations was explored following the Nanotechnology Characterization Lab (NCL) protocol for analysis of Haemolytic Properties of Nanoparticles (NCL ITA-1 Version 1.2), with slight modifications. Briefly, fresh human blood was drawn from healthy participants in the Laboratory of Clinical Analysis of the Faculty of Pharmacy of the University of Coimbra, after a written informed consent was obtained from all individuals. The venous blood, collected in heparinised vacutainer tubes, was properly diluted in PBS to final a concentration of 10 mg/mL of total blood haemoglobin, and incubated with the NLCs at eight different concentrations, regarding ATO content for 3 h at 37° C. Aqueous and acetate buffered polysorbate 80 solutions were also tested in 15 and 100 μg/mL. PBS and Triton™ X-100 were used as negative and positive controls, respectively, with additional no-blood controls being used due to possible interference of CUR at 540 nm. Following incubation, cell-free supernatants were obtained and any plasma-free haemoglobin and metabolites were converted into cyanmethaemoglobin (CMH), using Drabkin's reagent. The detection of CMH was conducted at 540 nm and the percentage of haemolysis (%) was calculated taking into account the equation 2.

$$\% Haemolysis = \frac{Abs sample ‐ Abs negative control ‐ Abs no ‐ blood control}{Abs positive control ‐ Abs negative control ‐ Abs no ‐ blood control} \times 100 \qquad ( eq . 2 )$$

### Example 4—In Vivo Biodistribution and Safety Evaluation Studies

Adult male CD-1 and CD-1 nude mice, aged between 6-7 weeks and weighting 25-40 g, were purchased from Charles River Laboratories (Lyon, France) and maintained in local animal facilities under controlled conditions (12 h of light/dark cycle, at 20±2° C. and 50±5% of relative humidity), with free access to standard diet and water. All animal experiments were conducted in agreement with the international regulations of the European Union.

F5 formulation was administered to mice by intraperitoneal route, in a single dose (40 mg/Kg of ATO and 20 mg/Kg of CUR). A solution containing both compounds in PEG 400:PBS (pH=7.4) (1:1, V/V) was used as control. Thereafter, mice were sacrificed by decapitation at predetermined post-dosing time points. Blood samples were collected to heparinised tubes and centrifuged at 2880 g for 10 min at 4° C. Plasma supernatant was collected and frozen at −80° C. until analysis. After exsanguination, the brain, liver and spleen were carefully excised and immediately weighted. Brain and liver tissues were homogenized with an H2O-acetonitrile (1:1, V/V) solution (4 mL per g of tissue) using a Thomas® Teflon pestle tissue homogenizer and centrifuged at 4 150 g for 15 min at 4° C. In parallel, spleen tissues were homogenized with 1 mL of the same solution using a high-speed stirrer (Ultra-Turrax X1020, Ystral GmbH, Dottingen, Germany) and then, centrifuged at 12 045 g for 5 min at 4° C. All supernatants were collected and stored at −80° C. until analysis using a previously validated HPLC method.

The maximum peak concentration (Cₘₐₓ) in plasma, brain, liver and spleen of ATO and CUR and the corresponding time to reach Cₘₐₓ (tₘₐₓ) were directly retrieved from the experimental data obtained. Taking into account the non-compartmental model and based on the mean concentration values for each time point, the remaining pharmacokinetic parameters were estimated with WinNon-Lin software (version 5.3, Pharsight Corporation, Mountain View, Calif., USA). The pharmacokinetic parameters regarding the administration of the NLC to mice are displayed in Table 2.

TABLE 2
Pharmacokinetic parameters obtained for co-encapsulated ATO and CUR in F0 and
F5, in comparison to free in solution, in plasma, brain, liver and spleen.

| | Atorvastatin calcium | | | | | | | | | | | |
|---|---|---|---|---|---|---|---|---|---|---|---|---|
| Pharmacokinetic | Control | | | | F0 | | | | F5 | | | |
| parameters | Plasma | Brain | Liver | Spleen | Plasma | Brain | Liver | Spleen | Plasma | Brain | Liver | Spleen |
| tₘₐₓ (min) | 20 | 15 | 15 | 10 | 30 | 30 | 10 | 10 | 20 | 20 | 10 | 10 |
| Cₘₐₓ | 11.91 | 0.37 | 52.14 | 33.02 | 8.87 | 0.38 | 68.78 | 48.70 | 4.05 | 0.24 | 41.37 | 23.55 |
| (μg/mL or μg/g) | | | | | | | | | | | | |
| AUC₀₋ₗₐₛₜ | 417.16 | 4.46 | 1464.2 | 1331.6 | 511.11 | 48.02 | 2943.8 | 1769.9 | 222.19 | 10.12 | 2220.1 | 1084.7 |
| (min × μg/mL or | | | | | | | | | | | | |
| min × μg/g) | | | | | | | | | | | | |
| AUCₐₗₗ | 417.16 | 5.15 | 1464.2 | 1331.6 | 578.25 | 48.02 | 2943.8 | 2680.4 | 300.79 | 15.13 | 2220.1 | 1448.1 |
| (min × μg/mL or | | | | | | | | | | | | |
| min × μg/g) | | | | | | | | | | | | |
| tₘₐₓ (min) | 5 | 5 | 5 | 5 | 10 | 10 | 10 | 10 | 20 | 120 | 10 | 20 |
| Cₘₐₓ | 1.59 | 0.14 | 0.35 | 1.87 | 1.30 | 0.02 | 2.34 | 3.95 | 0.39 | 0.08 | 0.54 | 1.32 |
| (μg/mL or μg/g) | | | | | | | | | | | | |
| AUC₀₋ₗₐₛₜ | 32.41 | 1.53 | 11.23 | 43.25 | 55.97 | 0.28 | 40.20 | 92.19 | 5.99 | 9.14 | 22.01 | 10.95 |
| (min × μg/mL or | | | | | | | | | | | | |
| min × μg/g) | | | | | | | | | | | | |
| AUCₐₗₗ | 32.41 | 1.53 | 11.23 | 43.25 | 60.05 | 0.33 | 43.40 | 136.34 | 6.98 | 9.14 | 22.01 | 17.53 |
| (min × μg/mL or | | | | | | | | | | | | |
| min × μg/g) | | | | | | | | | | | | |

tₘₐₓ—time to achieve the maximum concentration; Cₘₐₓ—Maximum concentration; AUC0-inf—area under the concentration time-curve from time zero to infinite; AUC₀₋ₗₐₛₜ—Area under the concentration time-curve from time zero to the last measurable drug concentration.

The drug selectivity index (DSI), which concerns the organ-to-plasma partitioning ratio of the drug administered by intraperitoneal injection to mice was estimated according to equation 3,

$$DSI = \frac{{AUC}_{organ}}{{AUC}_{plasma}} \qquad ( Eq . 3 )$$

where AUC_{organ} and AUCₚₗₐₛₘₐ correspond to the areas under the drug concentration-time curves for a determined organ and plasma, respectively. A DSI index higher than 1 indicates preferential selectivity of drug to the determined organ. A drug targeting index (DTI) that concerns the organ-to-organ partitioning ratio of the encapsulated compared to the non-encapsulated drug formulation, administered by intraperitoneal injection to mice was calculated according to equation 4,

$$DTI = {( \frac{{AUC}_{encapsulated}}{{AUC}_{non - encapsulated}} )}_{organ} \qquad ( Eq . 4 )$$

where AUC_{encapsulated} and AUC_{non-encapsulated} correspond to the areas under the drug concentration-time curves for a determined formulation, in the predefined organ. A DTI index larger than 1 indicates preferential targeting of the encapsulated drug to the determined organ. These results are presented in FIG. 5.

### Example 5—In Vivo Efficacy Study

Regarding orthotopic U-87 MG human GB tumor model, before the surgical procedure, fourteen nude mice were weighted, given buprenorphine (0.1 mg/Kg), ketamine (100 mg/Kg) and xylazine (10 mg/Kg) intraperitoneally and placed on a stereotactic frame. After the identification of the injection site, at the Cartesian coordinates (x,y,z)=(2.1,0.5,−3) in relation to bregma, approximately 36 000 U 87 MG cells were injected in the striatum of the frontal cortex of the right hemisphere of the brain, using a Hamilton syringe. In every mouse, the presence, location and volume of the tumors were assessed by Magnetic Resonance Imaging (MRI) weekly. MRI was performed using a 9.4T Bruker BioSpec 94/20USR system (Bruker, Germany) after anesthetizing mice with isoflurane (1 L/min, induction: 4% isoflurane; maintenance: 2% isoflurane). Vital signs, in particular, respiratory frequency and body temperature, were monitored continuously. For structural analysis, T2-weighted images were acquired in sagittal, coronal and axial planes, using a rapid acquisition with relaxation enhancement sequence (T2 Turbo RARE). Tumor images were manually segmented in the 3D Slicer software for 3D reconstruction, in order to calculate tumor volume.

At 10 days post-tumor inoculation, mice were randomly divided into three groups and treated daily with intraperitoneal injections of the F5 formulation (n=5), with a solution containing both drugs in PEG 400:saline (1:1, V/V) (n=4) or with saline (n=5). Similarly to the safety evaluation test, all animals were closely observed during the immediate post injection period. Mice physical appearance (body weight and coat condition), body function (food intake), presence of loose stools, diarrhea or blood in diarrhea and behavior (handling, aggression, abnormal gait and posture and reluctance to move) were also monitored throughout the study. The hepatic function was evaluated by quantifying the levels of AST, ALT and ALP before euthanizing the animals. Pre-determined human endpoints for animal euthanasia were also selected, specifically, a weight loss over 20% of initial body weight and lack of feeding or diarrhea for more than 48 h. Note that the use of PEG 400 may promote diarrhea, as it presents laxative properties. In the solution group, this endpoint was not taken into consideration. Tumor diameter and volume measures by Magnetic Resonance Imaging are introduced in FIG. 6, whereas body weight and hepatic function of tumor bearing mice are displayed in FIG. 7.

The disclosure should not be seen in any way restricted to the embodiments described and a person skilled in the art will foresee many possibilities to modifications thereof.

The above-described embodiments are combinable.

1. Drug delivery system for targeted therapy comprising a functionalized lipid-based nanoplatform, wherein at least one ligand is coupled to the surface of the nanoplatform and encapsulates at least one pharmaceutically active ingredient.

2. Drug delivery system according to claim 1 for brain tumor therapy, wherein the ligand is a tumor-specific ligand.

3. Drug delivery system according to claim 1 for treatment of brain tumors, particularly glioblastoma.

4. Drug delivery system according to claim 1, wherein the lipid-based nanoplatform is a lipid matrix comprising a lipid content of 5-20% (w_{lipid}/wₜₒₜₐₗ).

5. Drug delivery system according to claim 1, wherein the lipid-based nanoplatform comprises a liquid lipid fraction and a solid lipid fraction, particularity wherein the solid lipid fraction:liquid lipid fraction ratio is from 25:75 to 75:25.

6. (canceled)

7. Drug delivery system according to claim 1, wherein the solid lipid fraction:liquid lipid fraction ratio is 25:75 to 75:25 and the solid lipid fraction comprises cetyl palmitate.

8. Drug delivery system according to claim 1, wherein the nanoplatform comprises one or more solid lipids selected from: stearic acid, glyceryl tripalmitate, cetyl alcohol, cetostearyl alcohol, mono-, di-, triglyceride esters of fatty acids C10-C18, mono-, di-, triesters of C16-C22 acids or a combination thereof; or one or more liquid lipids selected from: caprylocaproyl macrogol-B glycerides, diethylene glycol monoethyl ether or a combination thereof, propylene glycol mono-, diesters of C8-C12 acids, medium-chain mono-, di-, triglycerides of C8-C10, monounsaturated omega-9 octadecenoic fatty acid, C30 isoprenoid hydrocarbon, hydrogenated C30 hydrocarbon or a combination thereof.

9. (canceled)

10. (canceled)

11. Drug delivery system according to claim 1, wherein the nanoplatform is a lipid matrix additionally comprising a non-ionic surfactant, preferably wherein the non-ionic surfactant is fat free soybean phospholipids at 0.5-3% (w/w), polysorbate 80 at 1-5% (w/w) or a combination thereof, more preferably wherein the non-ionic surfactant is one or more selected from: fat free soybean phospholipids, phosphatidylcholine, polysorbate 80, polysorbate 60, polysorbate 40, polysorbate 20, soybean phosphatidylcholine, soybean hydrogenated phosphatidylcholine, polyethylene glycol (15)-hydroxystearate, polyoxyl 40 hydrogenated castor oil, purified polyoxyl 35 castor oil or a combination thereof.

12. (canceled)

13. (canceled)

14. Drug delivery system according to claim 1, further comprising a cationic surfactant, preferably wherein the cationic surfactant is octadecylamine, more preferably wherein the cationic surfactant is one or more selected from: cetrimonium bromide, dodecyltrimethylammonium bromide, didodecyldimethylammonium bromide dodecyl pyridinium chloride of a combination thereof.

15. (canceled)

16. (canceled)

17. Drug delivery system according to claim 1, wherein the mean particle size of the lipid-based nanoplatform is lower than 200 nm, preferably 50 to 150 nm.

18. Drug delivery system according to claim 1, wherein the nanoplatform has a monodisperse size distribution with a polydispersity index lower than 0.3, preferably from 0.1 to 0.3.

19. Drug delivery system according to claim 1, wherein the nanoplatform comprises a nanostructured lipid carrier, preferably a nanostructured lipid carrier comprising at least one pharmaceutically active ingredient.

20. (canceled)

21. Drug delivery system according to claim 1, wherein the nanostructured lipid carrier comprises at least one pharmaceutically active ingredient and the pharmaceutically active ingredient is encapsulated, entrapped or intercalated in the nanostructured lipid carrier.

22. Drug delivery system according to claim 1, wherein the pharmaceutically active ingredient is one or more selected from the group consisting of alkylating drugs; cytotoxic antibiotics; platinum drugs; antiparasitics; anti-infectives; drugs acting on nervous system; drugs acting on cardiovascular system; drugs acting on blood; drugs acting on alimentary tract and metabolism; drugs acting on genitourinary system and sex hormones; drugs acting on musculoskeletal system; drugs acting on respiratory system, dermatological drugs or a combination thereof; wherein the alkylating drug is one or more selected from: temozolomide, chlorambucil, melphalan, busulfan, lomustine, carmustine, estramustine, cyclophosphamide, chlormethine, treosulfan, mitobronitol or a combination thereof; wherein the cytotoxic antibiotic is one or more selected from: doxorubicin, epirubicin, idarubicin, daunorubicin, aclarubicin, dactinomycin, mitomycin, bleomycin, mitoxantrone or a combination thereof; wherein the platinum drug is one or more selected from: cisplatin, carboplatine, oxaliplatin or a combination thereof; wherein the antiparasitic is one or more selected from: chloroquine, hydroxychloroquine, mefloquine, quinacrine, pyrvinium pamoate, mebendazole or a combination thereof; wherein the anti-infective is one or more selected from: acyclovir, valganciclovir, lopinavir, ritonavir, nelfinavir, atazanavir, ribavirin, itraconazole, ciprofloxacin, salinomycin, minocycline, doxycycline, tigecycline, chloramphenicol or a combination thereof; wherein the drug acting on nervous system is one more selected from: chlorpromazine, thioridazine, fluphenazine, perphenazine, olanzapine, penfluridol, quetiapine, lithium, donepezil, memantine, paroxetine, fluoxetine, sertraline, fluvoxamine, imipramine, amitriptyline, clomipramine, doxepin, citalopram, escitalopram, levetiracetam, valproic acid, propofol, disulfiram, dimethyl fumarate or a combination thereof; wherein the drug acting on cardiovascular system selected from: digitoxin, atorvastatin, lovastatin, simvastatin, mevastatin, fluvastatin, cerivastatin, pitavastatin, verapamil, miberfradil, losartan, captopril, carvedilol or a combination thereof; wherein the drug acting on blood is ticlonidine; wherein the drug acting on alimentary tract and metabolism is one or more selected from: metformin, repaglidine, pioglitazone, rosiglitazone, ciglitazone, phenformin, sulfasalazine, aprepitant, cimetidine or a combination thereof; wherein the drug acting on genito urinary system and sex hormones one or more selected from: estradiol, sildenafil or a combination thereof; wherein the drug acting on musculoskeletal system and sex hormones is one or more selected from: auranofin, celecoxib or a combination thereof; wherein the drug, acting on respiratory system is one or more selected from: ibudilast, amlexanox or a combination thereof; wherein dermatological drug is one or more selected from: isotretinoin, ivermectin or a combination thereof.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. Drug delivery system according to claim 1, wherein the ligand is selected from: hyaluronic acid, folic acid, cRGDfK peptide, H7k(R₂)₂ peptide or a combination thereof.

37. Drug delivery system according to the previous claim wherein folic acid, cRGDfK peptide and H7k(R₂)₂ peptide are coupled to hyaluronic acid.

38. Drug delivery system according to claim 1 for targeting a molecule overexpressed in tumor cells and/or tumor blood vessels.

39. Process for obtaining the drug delivery system of claim 1 comprising the steps of high shear homogenization followed by ultrasonication or high-pressure homogenization.

40. Intravenous composition comprising the drug delivery system of claim 1.
